(19) United States
(12) Patent Application Publication
(10) Pub. No.: US 2013/0190586 A1
(43) Pub. Date: Jul. 25, 2013

(54) Multi-Terminal Nanoelectrode Array

(71) Applicants: A. George Akingba, Carmel, IN (US); Peng-Sheng Chen, Indianapolis, IN (US); Aamer Mahmood, West Lafayette, IN (US)

(72) Inventors: A. George Akingba, Carmel, IN (US); Peng-Sheng Chen, Indianapolis, IN (US); Aamer Mahmood, West Lafayette, IN (US)

(73) Assignee: Indiana University Research & Technology Corporati, Indianapolis, IN (US)

(21) Appl. No.: 13/877,210

(22) PCT Filed: Oct. 6, 2011
(86) PCT No.: PCT/US2011/055103

§ 371 Date: Apr. 1, 2013

Related U.S. Application Data
(60) Provisional application No. 61/390,541, filed on Oct. 6, 2010.

(51) Int. Cl. A61B 5/04; A61N 1/05

(52) U.S. Cl. CPC A61B 5/04001; A61N 1/0551
USPC 600/372; 607/116

(57) ABSTRACT

An electrode for monitoring nerve activity has been developed. The electrode includes an array of electrically conductive projections extending from a surface of an electrical contact that enable the electrical contact to be connected directly to the nerve.

### GOVERNMENT INTEREST

This invention was made with government support under grant number HL071140 awarded by the National Institutes of Health (NIH). The United States government has certain rights in the invention.

### TECHNICAL FIELD

The present disclosure relates generally to a device for measuring electrical activity in nerves, and for applying electrical stimulation to nerves. In particular, the disclosure relates to electrodes for measuring sympathetic nerve activity and for applying electrical stimulation to the sympathetic nerves.

### BACKGROUND

Many diagnostic and treatment methods in the fields of medicine and biology rely on measurements of nervous activity in patients and test subjects. Nervous activity in humans and other animals generates electrical signals that are detectable by electronic equipment such as oscilloscopes and other electrical signal processing devices. In order to detect the nerve activity, one or more electrical conductors, or electrodes, are placed in proximity to the nerves being measured. The electrodes may receive the electrical signals for further medical analysis. Various medical treatment methods also use electrodes to deliver electrical signals to the nerves in order to induce a response in the patient.

Cardiac care is one particular area of medical treatment that heavily utilizes measurement of nerve activity. Activity in the autonomic nervous system controls the variability of the heart rate and blood pressure. The sympathetic and parasympathetic branches of the autonomic nervous system modulate cardiac activity. Elevated levels of sympathetic nerve activity (SNA) are known to be correlated with heart failure, coronary artery disease, and may be associated with the initiation of hypertension. Therefore, a diagnostic index of “autonomic tone” produced in accordance with measurement of SNA may have considerable clinical value. As known in the art, clinical utilization of autonomic nervous activity is mostly derived from biochemical perturbations like the use of beta-blockers in high blood pressure management. While elevated levels of SNA are known to be correlated with these medical conditions, more precise analysis of the particular electrical signals produced by sympathetic nerves is needed before sympathetic nerve measurement can become a useful diagnostic or prognostic tool. Deficiencies in current electrode technology result in either poor autonomic signal quality or present some difficulty in integrating implantable electronic enhancements (like telemetry, on-chip amplification, storage memory, and motion sensors).

One challenge to measuring sympathetic nerve activity is that the magnitude of electrical signals in the sympathetic nerves is relatively low, while various other electrical signals present in the patient provide noise that may interfere with isolation and detection of the sympathetic nerve activity. Existing electrodes detect both the nerve activity and other electrical noise generated in the patient's body. Thus, the signal to noise ratio (SNR) of the sympathetic nerve activity measured using electrodes known to the art is low, hindering the accurate detection and characterization of sympathetic nerve activity. For example, known electrodes have measured nerve signals with a voltage of 35 μV while the level of noise in the measuring electrode is 10 μV. Using the following SNR equation

$$SNR = 20 log ( \frac{{V}_{signal}}{{V}_{noise}} ) dB ,$$

the example signals have an SNR of approximately 10.9 dB. While this signal to noise ratio permits some measurements of relatively large changes in sympathetic nerve activity, the noise level may mask nerve activity having a smaller voltage magnitude. Improvements to electrodes that increase the accuracy of nerve activity measurement, including sympathetic nerve activity measurement, will benefit the fields of medicine and biology.

### SUMMARY

An electrode for measuring nerve activity has been developed. The electrode includes a first electrical contact having at least one electrically conductive projection extending from a surface of the first electrical contact, and a first electrical lead electrically connected to the first electrical contact to enable signals from the nerve to be received. The at least one electrically conductive projection is configured to engage tissue proximate to at least one nerve to enable the first electrical contact to electrically contact the nerve directly and form an electrically conductive or inductive path between the nerve and the electrical contact.

### BRIEF DESCRIPTION OF THE DRAWINGS

FIG. 1A is a cross-sectional view of a two-terminal electrode array.

FIG. 1B is a cross-sectional view of a three-terminal electrode array.

FIG. 2 is a photograph of a microscopic view of an array of electrical probes formed in an electrode array.

FIG. 3 is a front view of an embodiment of the two-terminal electrode array of FIG. 1A.

FIG. 4 is a flow diagram of a process for forming arrays of nanoelectrode tips for use with an electrode.

FIG. 5A is a cross-sectional diagram of a silicon wafer with a top and a bottom silicon oxide layer formed on either side of the wafer.

FIG. 5B is a diagram of the silicon wafer of FIG. 5A with a mask layer formed the top silicon oxide layers.

FIG. 5C is a diagram of the silicon wafer of FIG. 5B after an etching process forms pillars in the silicon wafer.

FIG. 5D is a diagram of the silicon wafer of FIG. 5C with silicon probes formed from the pillars extending from the silicon wafer.

FIG. 5E is a diagram of the silicon wafer of FIG. 5D with the silicon probes exposed.

FIG. 5F is a diagram of the silicon wafer of FIG. 5E with a dielectric layer formed over the silicon probes and the silicon wafer.

FIG. 5G is a diagram of the silicon wafer of FIG. 5F with a mask applied to the bottom dielectric layer.

FIG. 5H is a diagram of the silicon wafer of FIG. 5G after an etching process removes unmasked portions of the bottom dielectric, silicon oxide, and silicon wafer including the silicon probes.

FIG. 5I is a diagram of the silicon wafer of FIG. 5H after vapor deposition of a metal to form electrically connected metallic probes.

FIG. 5J is a diagram view of the silicon wafer of FIG. 5I including two separate electrical probes connected to electrical leads.

FIG. 6 is a prior art diagram of a nerve surrounded by an epineurium layer.

### DETAILED DESCRIPTION

The description below and the accompanying figures provide a general understanding of the environment for the system and method disclosed herein as well as the details for the system and method. In the drawings, like reference numerals are used throughout to designate like elements. As used herein, the term “electrode” refers to an electrical conductor that is configured to establish an electrical contact with biological tissue such as tissue in a patient or test subject. As used herein, the term “nanoelectrode tip” or “nanoelectrode probe” refers to an electrically conductive electrode probe or needle having a size and shape that enables the nanoelectrode tip to engage a layer of tissue to establish electrical contact with a nerve. The nanoelectrode probes can be formed in various sizes and configurations, with typical sizes of an individual nanoelectrode probe being microscopic. Despite the use of the term “nano,” nanoelectrode probes can be larger than one nanometer and are often several hundred or thousand nanometers long and are tens or hundreds of nanometers in diameter at the tip of the probe.

The terms “nanoelectrode array” or “electrode array” both refer to a plurality of nanoelectrode tips that are electrically connected to one another and arranged in a predetermined pattern. The term “two-terminal nanoelectrode array” refers to an electrode having two electrical contacts where at least one of the contacts is a nanoelectrode array. The term “three-terminal nanoelectrode array” refers to an electrode having three electrical contacts where at least one of the contacts is a nanoelectrode array. As used herein, the term “wafer” refers to a planar material sheet adapted to have multiple repeated instances of a structural pattern formed on and through the surface of the wafer. A common example of a wafer is a silicon wafer used in the fabrication of microelectronic devices. Common examples of these wafers have approximately circular shapes with diameters between 25 mm and 450 mm and thicknesses of approximately 275 μm to 950 μm. While the wafer is often primarily composed of a silicon substrate, wafers may also include planar layers of other materials, such as metals and dielectrics.

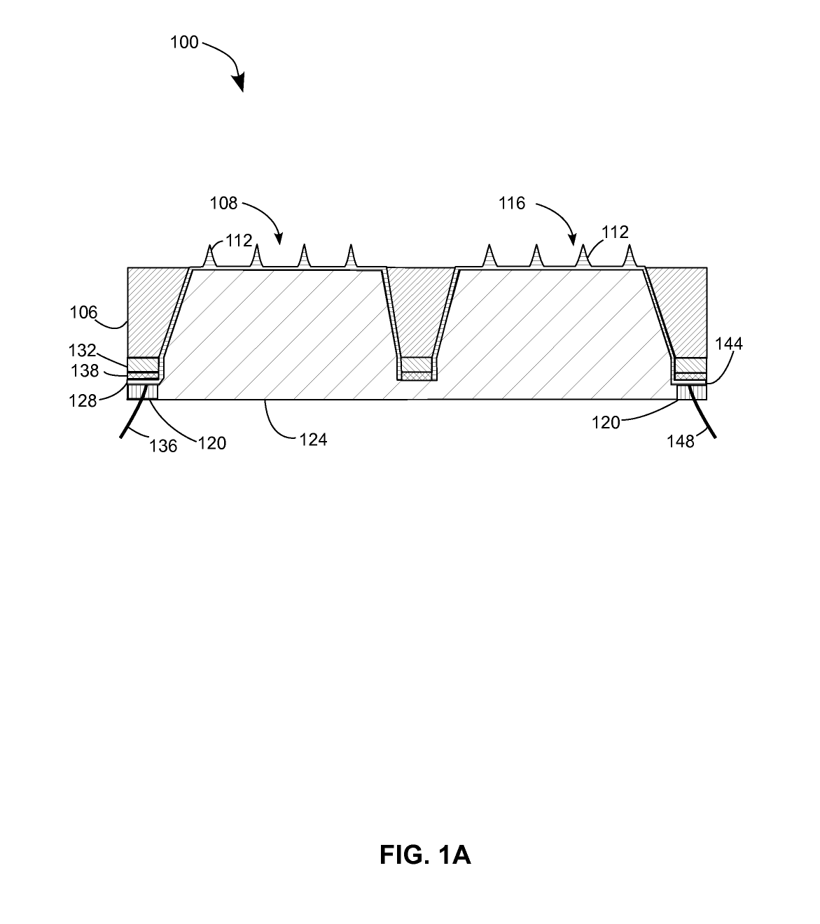

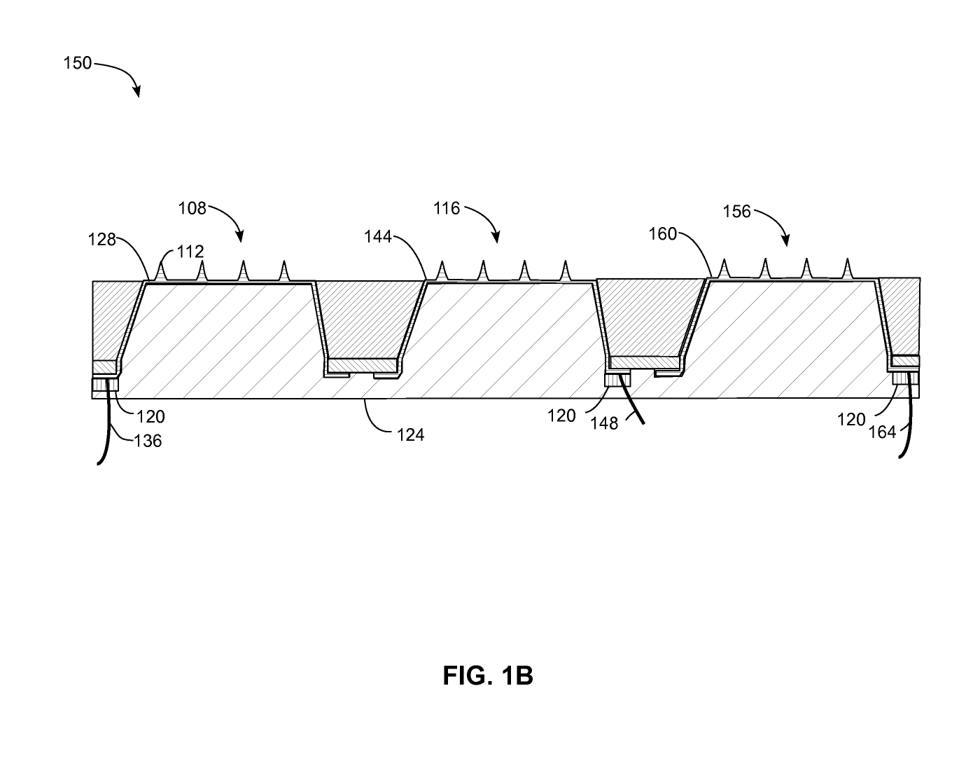

FIG. 1A depicts an exemplary two-terminal nanoelectrode array 100. The two-terminal nanoelectrode array 100 includes two nanoelectrode arrays 108 and 116, electrically conductive layers 128 and 144, silicon layer 106, silicon oxide layer 132, dielectric layer 138, electrically conductive adhesive 120, electrically insulating adhesive 124, and electrical lead wires 136 and 148. The nanoelectrode arrays 108 and 116 each include a plurality of nanoelectrode tips, such as tip 112, which extend from the surface of each electrode array. The silicon layer 106 and silicon oxide layer 132 electrically isolate the nanoelectrode arrays 108 and 116. The silicon layer 106 is formed from a high resistivity silicon that resists a flow of electrical current between the electrically conductive layers 128 and 144. Each nanoelectrode tip 112 is substantially composed of gold or another electrical conductor that is fully exposed to contact nerve tissue for measuring nerve signals.

Each of the nanoelectrode arrays 108 and 116 includes an electrically conductive layer 128 and 144, respectively. The electrically conductive layers include bonding pads for establishing an electrical connection to one of wires 136 and 148. The nanoelectrode arrays 108 and 116 and the electrical conductors are both formed from single layer of metal in some embodiments. In one embodiment, the electrically conductive layers 128 and 144 are formed from the same material as each nanoelectrode, such as gold, and promote a uniform electrical contact between each of the nanoelectrode tips and the electrical leads.

The electrically insulating adhesive 124 seals openings formed through the silicon layer 106, silicon oxide layer 132, and dielectric layer 138 to prevent fluids, tissue, or other contaminants from a patient or the environment surrounding the electrode 100 from contacting the back side of either of the nanoelectrode arrays 108 and 116. In some configurations, the electrically insulative adhesive 124 does not completely fill the space under the nanoelectrode arrays 108 and 116, but seals an air pocket under each of the nanoelectrode arrays 108 and 106. Suitable adhesive materials include a silicone elastomer with a resistivity of 1.8×10¹⁵ Ω·cm and electrically insulative epoxies. One commercially available silicone elastomer is Dow Corning® 3745 RTV sold by the Dow Corning Corporation of Midland, Mich., USA. The electrically insulating adhesive 124, silicon layer 106, and silicon oxide layer 132 electrically isolate the nanoelectrode arrays 108 and 116. Thus, electrical nerve signals generated in the nerve tissue are conducted through the nanoelectrode arrays 108 and 116 through the leads 136 and 148, respectively, and do not form a circuit between the nanoelectrode arrays 108 and 116.

The electrical leads 136 and 148 may be formed from any electrically conductive material suited for use in a medical environment, including copper wires surrounded by an insulated jacket. Remote ends of wires 136 and 148 may connect to a variety of medical diagnostic equipment, including wireless transmitters embedded in the body of a patient. Additionally, the wires may connect to electrical signal generators for application of electrical stimulation to various nerves.

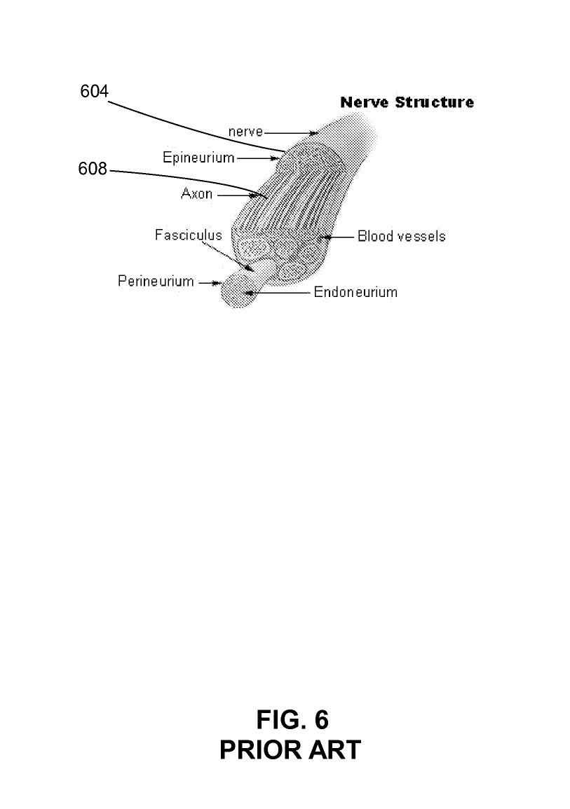

In operation, the two-terminal nanoelectrode array 100 is placed in contact with tissue of a patient or test subject proximate to a nerve undergoing measurement or electrical stimulation. The nanoelectrode tips in nanoelectrode arrays 108 and 116 can detect electrical signals in the nerve tissue in two different ways. First, the nanoelectrode tips penetrate a layer of tissue that is proximate to a nerve while not damaging the nerve. As shown in FIG. 6, one such layer of tissue is the epineurium layer 604 that surrounds various peripheral nerves including axons 608 of sympathetic nerves. Both of the nanoelectrode arrays 108 and 116 penetrate the surrounding tissue and establish low electrical resistance contact with nerve cells. Second, the nanoelectrode arrays 108 and 116 can detect electrical signals through induction. The electrical activity in the nerve tissue generates an electrical field that induces a current in each of the nanoelectrode arrays. In the two-terminal configuration of nanoelectrode array 100, each of the nanoelectrode arrays 108 and 116 detects a separate electrical signal in the nervous tissue. A differential amplifier, such as a differential operational amplifier or other detector, generates a signal corresponding to a difference between the voltages generated in each of the nanoelectrode arrays 108 and 116 for use with signal detection and medical diagnostic equipment.

A system that measures electrical activity in nervous tissue of a patient can also detect spurious electrical signals, referred to as noise, from other sources than the nerve tissue. Sources of noise include diagnostic equipment connected to the terminals and external electromagnetic signals that generate noise in the electrical leads attached to the terminals. Various techniques known to the art can mitigate some external sources of noise. One source of noise, referred to as the Johnson noise also called Johnson-Nyquist noise, Nyquist noise or thermal noise, is electronic noise generated by the thermal agitation of charge carriers in an electric conductor and occurs regardless of any applied voltage. The Johnson noise level in an electrical circuit can be expressed as a voltage Vⱼ and is expressed with the following equation:

Vⱼ=\sqrt{4k_{b}RTΔf}

Where k_{b} is Boltzmann's constant, R is the resistance of the circuit (including and dominated by the resistance of the electrodes inserted for measurements in applications like this one) in Ohms, Δf is the frequency bandwidth in Hz of the signal at the terminal and T is the temperature in degrees Kelvin. In a practical situation, the body temperature of a patient provides the temperature T. Additionally, narrowing frequency bandwidth Δf can reduce noise, but leads to a loss of information in the nerve signal being measured by the nanoelectrode. The nanoelectrode terminals in the nanoelectrode array 100 establish electrical contacts with nerves over a broad surface area that have a lower electrical resistance between the nerves and the electrode than electrodes previously known in the art. The reduction of the resistance R also reduces the magnitude of noise voltage Vⱼ and consequently reduces measured noise when measuring nerve activity, without narrowing the frequency bandwidth Δf. The reduction in noise results in improved signal to noise ratios when measuring nerve activity, including sympathetic nerve activity. Thus, the structure of the terminals in the nanoelectrode array 100 enable improved detection of electrical nervous activity over prior art devices.

FIG. 1B depicts an example of a three-terminal nanoelectrode array 150. Three-terminal nanoelectrode array 150 includes nanoelectrode arrays 108 and 116, metal layers 128 and 144, oxide layer 106, electrically conductive adhesive 120, electrically insulating adhesive 124, and electrical leads 136 and 148 as shown in FIG. 1A. The three-terminal nanoelectrode array 150 also includes a third nanoelectrode array 156 with an associated metal layer 160 and wire 164. Nanoelectrode array 156 is bonded to wire 164 with the conductive adhesive 120 in a similar manner to nanoelectrode arrays 108 and 116. Three-terminal nanoelectrode array 150 operates in a similar manner to two-terminal nanoelectrode array 100, with the three nanoelectrode arrays enabling simultaneous measurement of nerve activity and electrical stimulation of nervous tissue. For example, nanoelectrode array 116 may act as a reference electrode with nanoelectrode array 108 being used to receive electrical signals and enable differential voltage analysis of the nerve signals. The nanoelectrode arrays 108 and 116 detect electrical signals in the nerve tissue, and the nanoelectrode array 150 transmits electrical stimulation signals to the nerve. While two-terminal and three-terminal electrodes that include nanoelectrode arrays are exemplified herein, the nanoelectrode arrays such as arrays 108, 116, and 156 can be incorporated in electrodes having any number of terminals.

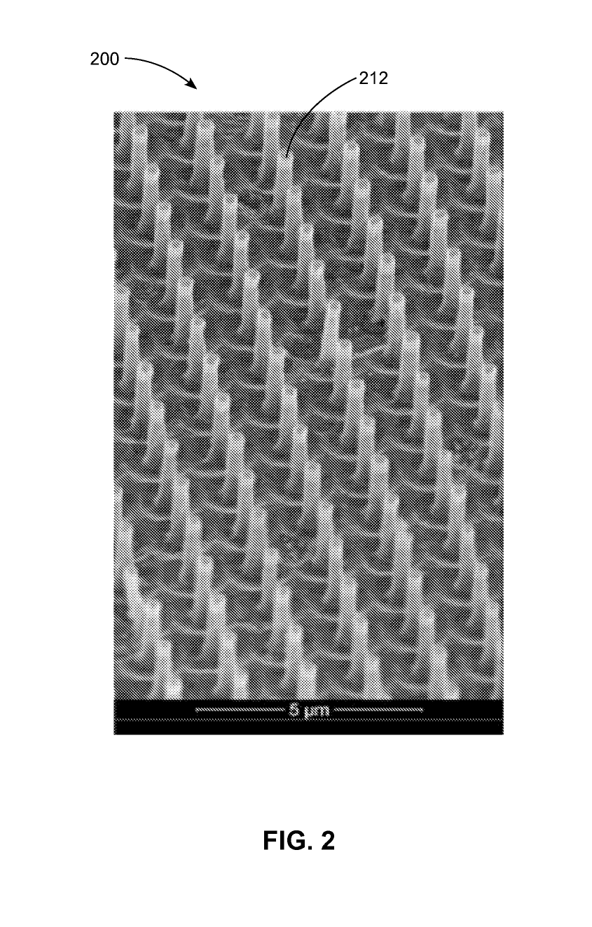

The arrays of nanoelectrode tips depicted in FIG. 1A-FIG. 1B are merely exemplary of one configuration of nanotips and are simplified for illustrative purposes. Alternative embodiments include electrodes with arrays of nanotips numbering in the tens or hundreds of thousands. FIG. 2 is a photograph of a microscopic view of a nanotip array 200. The nanotip array 200 includes nanotips 212 arranged in rows and columns over the surface of an electrode. The nanotip array 200 and nanotips 212 are formed from gold in one embodiment, but other conducting materials including titanium or other metals can be used to form the array. In the example of FIG. 2, each of the nanotips 212 has a height of approximately 2 μm and a diameter at the tip of approximately 250 nm. The nanotips 212 are arranged in rows and columns at approximately 1 μm intervals in the nanotip array 200. Alternative nanoelectrode configurations include nanotips with different dimensions and densities.

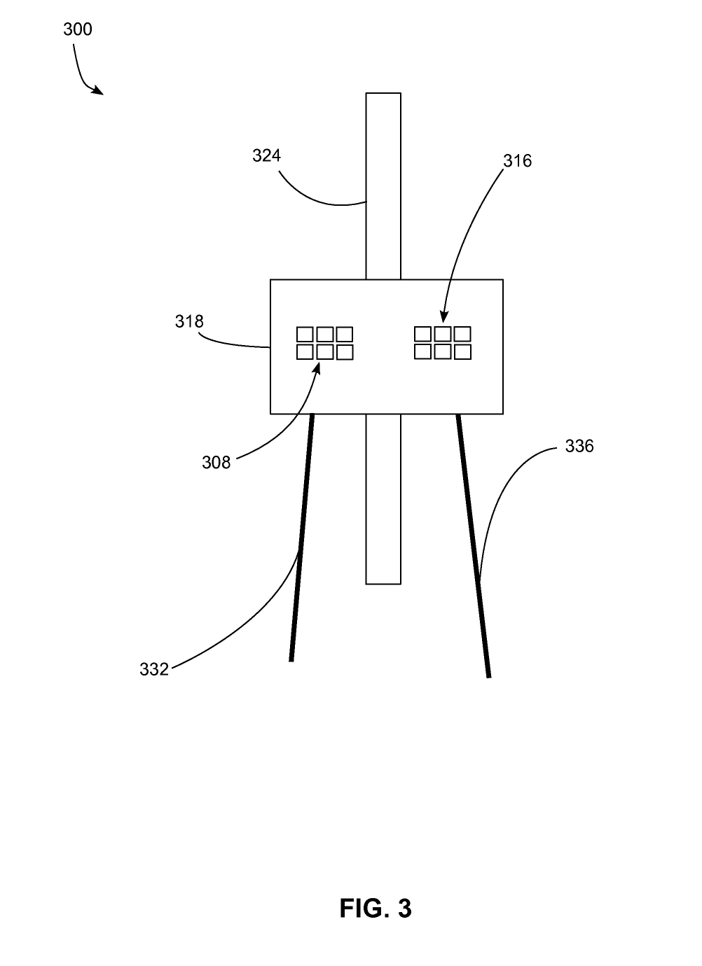

FIG. 3 is a macroscopic depiction of an exemplary electrode 300 having two terminals. The electrode 300 includes terminals 308 and 316 that each include nanoelectrode arrays. In the example of FIG. 3, the terminals 308 and 316 each form a single terminal that includes six separate nanoelectrode arrays. Each of the nanoelectrode arrays includes a metallic pad having an array of nanotips that engage nerve tissue in a patient. The six nanoelectrode arrays in each of the terminals 308 and 316 are electrically connected in parallel to form the two terminals 308 and 316 that each include six nanoelectrode arrays. A housing 318 holds the terminals 308 and 316. Two wires 332 and 336 extend from the housing 318 and are electrically connected to the terminals 308 and 316, respectively. The wires 332 and 336 are connected to, for example, an oscilloscope or other device that measures nerve activity in a patient. While each terminal 308 and 316 includes six nanoelectrode arrays in the electrode 300, alternative electrodes can include terminals with a different number of nanoelectrode arrays and nanoelectrode arrays having different sizes.

In the example of FIG. 3, the housing 318 is adhered to a Kapton strip 324. In operation, the nanoelectrode arrays 308 and 316 are placed in contact with nerve tissue in a patient, and sutures applied to the Kapton strip 324 hold the electrode 300 in place to enable continuous monitoring of nerve activity in the patient. Alternative configurations can use a different mounting material or structure to secure the electrode 300 in place with a nerve in the patient. The electrode 300 is small enough to enable surgical insertion of the electrode 300 into tissue of the patient. In one embodiment, the housing 318 has a width of approximately 4 mm, a height of approximately 2.5 mm, and a thickness of approximately 2-3 mm. Alternative embodiments of the electrode 300 can have different dimensions and can also include a three-terminal configuration that provides electrical stimulation to a nerve tissue in addition to monitoring electrical activity in the nerve tissue.

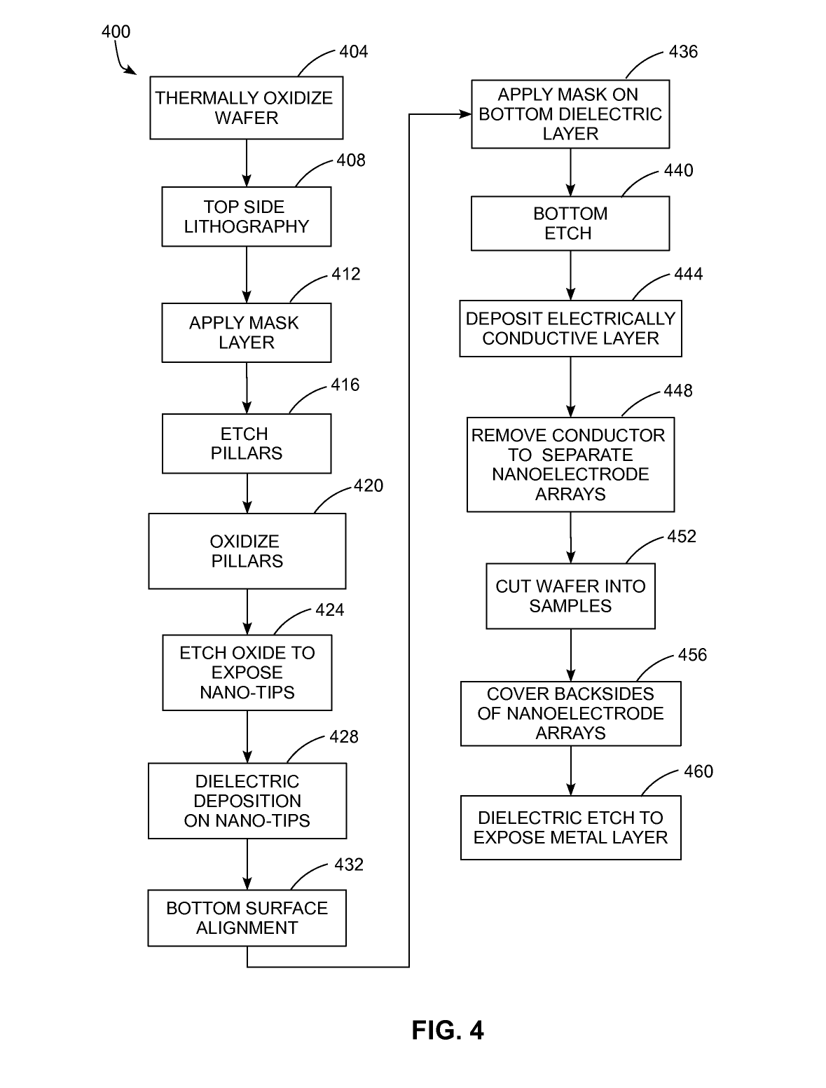

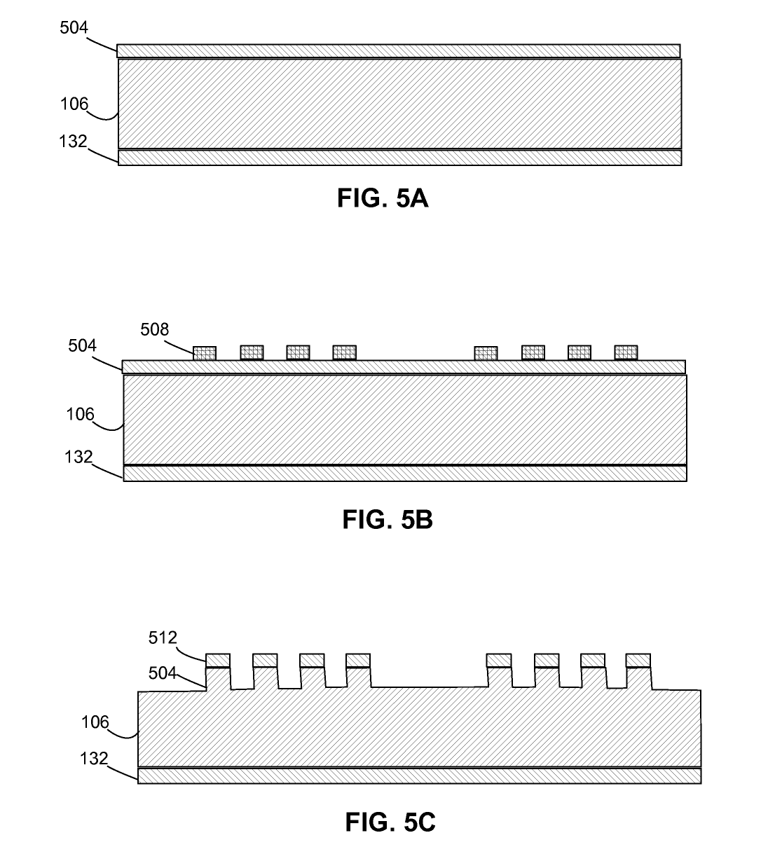

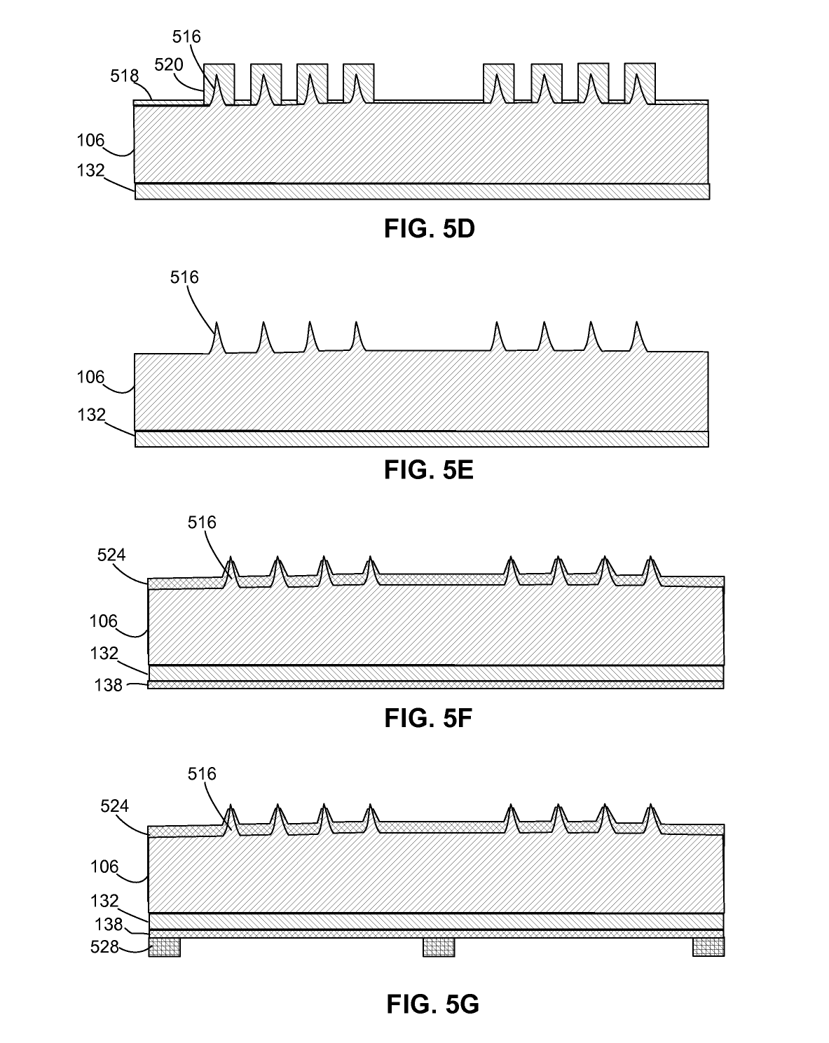

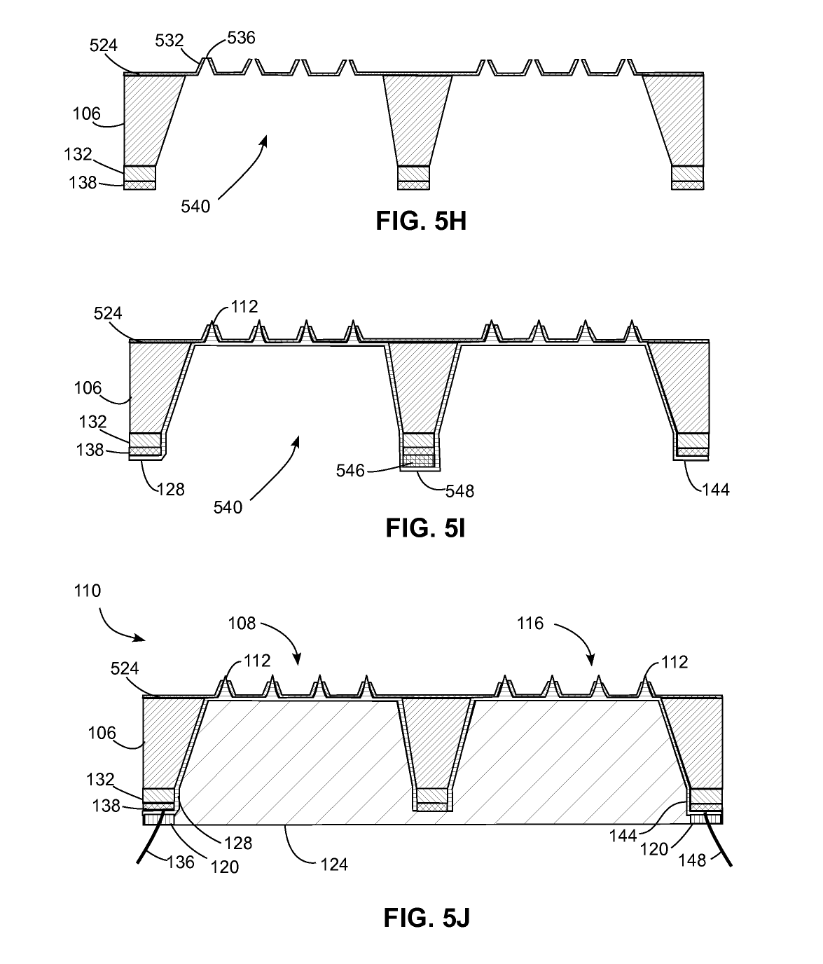

FIG. 4 is a block diagram of a process 400 for fabricating nanoelectrode arrays from a silicon wafer. FIG. 4 is described in conjunction with FIG. 5A-FIG. 5J and FIG. 1A that depict the structure of the silicon wafer during various stages of the fabrication process. Process 400 begins with thermal oxidation of a silicon wafer (block 404). As depicted in FIG. 5, the oxidizing process forms a top layer of silicon oxide 504 and bottom layer of silicon oxide 132 on the silicon wafer 106.

A lithography process, including electron beam lithography, optical lithography or another suitable lithography process, forms a pattern on the top silicon oxide layer 504 (block 408). Next, a mask layer of a resist material is formed on the pattern formed in the top surface of the wafer (block 412). The mask layer may be formed from a metal placed on the wafer using a lift-off technique. The mask layer is formed in locations of nanotips in the completed nanoelectrode array. FIG. 5B depicts a mask layer 508 selectively applied to the top silicon oxide layer 504.

Once the mask is in place, a reactive ion etch removes the unmasked portion of the top silicon oxide layer 504 and a portion of the silicon wafer 106 to form a series of silicon pillars under the masked portion of the top silicon oxide layer 504 and silicon wafer 106 (block 416). In one embodiment, each pillar includes a silicon pillar 504 that is approximately 2 μm tall, with a 500 nm thick silicon oxide top layer 512. The mask material 508 is removed from the tops of the pillars after completion of the etching process. FIG. 5C depicts the pillars including the top silicon oxide layer 512 and silicon 504. The pillars are formed in the pattern of the nanoelectrode tips in the nanoelectrode array.

After forming the pillars 504, the wafer is oxidized to convert a portion of the silicon 504 in each pillar into silicon oxide and leave a nanotip shaped segment of silicon 516 in each pillar (block 420). FIG. 5D depicts a silicon nanotip form 516 that is within a silicon oxide layer 520. The silicon oxide layer 520 includes the top silicon layer 512 of the pillar and a portion of the silicon pillar 504 that oxidizes during the oxidation process. The oxidation process leaves silicon nanotips 516 with shapes that correspond to the shapes of the metallic nanotips 112 in the completed electrode. The top layer of silicon oxide 512 in each pillar acts as a seed that accelerates the oxidation process near the top of each of the silicon pillars 504. Consequently, a greater portion of the silicon pillar 504 oxidizes near the top of the pillar, and the nanotips 516 have a broader silicon base that tapers to the narrower top of each nanotip 516. The height and sharpness of the silicon nanotips 516 can be controlled by adjusting the length of the oxidation process. The oxidation process also oxidizes a thin layer 518 of the top surface of the silicon wafer 106.

Process 400 applies a buffered oxide etch (BOE) solution to the top of the silicon wafer 106 to remove the silicon oxide 520 surrounding each of the silicon nanotips 516 and the silicon oxide layer 518 (block 424). FIG. 5E depicts the resulting wafer 106 with silicon nanotips 516. A low-pressure chemical vapor deposition process (LPCVD) forms an electrically non-conductive dielectric material over and between the silicon nanotips 516, forming the cap layer 524 depicted in FIG. 5F (block 428). Various electrically non-conductive dielectric materials used in the cap layer 524 include silicon nitride, silicon boride, and polymers such as Parylenes (p-xylylene polymers). In one embodiment, the cap layer 524 is approximately 1,000 Å thick. The vapor deposition process also deposits a second layer of the dielectric 138 on the bottom silicon oxide layer 132. In some embodiments, the cap layer 524 is thin enough that portions of the cap layer near the tops of the silicon nanotips 516 break, and the top of the silicon nanotips are exposed after deposition of the cap layer 524. In other embodiments, the cap layer 524 fully covers the silicon nanotips 516.

Process 400 continues by applying etching and lithography to the bottom silicon oxide layer 132 of each sample, which is also referred to as the backside of the wafer 106. Each sample is aligned from the bottom to facilitate etching and lithography from the bottom (block 432). A second mask is applied to the bottom layer of dielectric 138 (block 436), as depicted by the mask layer 528 in FIG. 5G.

Process 400 next removes the unmasked portions of the bottom silicon oxide layer 132 and portions of the silicon layer 106 including the silicon nanotips 516 with a second wet-etching process (block 440). The second wet-etching process includes two stages. The first stage removes unmasked portions of the bottom dielectric layer 138. The second stage removes unmasked portions of the bottom silicon oxide layer 132 and silicon wafer 106, but does not remove the dielectric cap 524. FIG. 5H depicts the wafer 106, bottom silicon oxide layer 132, and the cap 524 after the second etching process. The cap layer 524 includes hollow forms 532 that act as a mold for formation of metallic nanotips. In some embodiments, each form 532 includes an opening 536 that enables metallic nanotips to extend through the dielectric cap 524. The second etching process forms a cavity 540 through the bottom silicon oxide layer 132 and silicon wafer 106.

Process 400 forms the nanotips 112 using a deposition process applied to the bottom surface of the silicon oxide layer 132, silicon wafer 106, and cap layer 524 in the cavity 540 (block 444 ). The deposition process forms a layer of an electrical conductor, such as a metal or other electrically conductive material. The embodiment of FIG. 5I, a metal deposition process fills the hollow portion of each nanotip form 532 in the cap layer 524 to form the metallic nanotips 112, and also deposits a continuous metallic layer along the interior of the cavity formed in the sample. The continuous metal layer electrically connects all of the nanotips 112 in a single nanotip array to each other. As described above, gold is an appropriate metal for use in the metal layer, although other metals including titanium and other electrically conductive materials may be used as well. The metal layer is applied using a deposition process known to the art such as physical vapor deposition, including sputtering, evaporation, or chemical vapor deposition.

Prior to the metallization process, a resist layer 546 is placed on selected portions of the bottom silicon oxide layer 132 using lithographic techniques. After deposition of the metalusing a physical vapor deposition technique, such as evaporation or sputtering, a lift-off chemical process involving a photoresist stripper like the PRS-2000™ or a solvent like Acetone is used to remove the resist material 546 and the metal layer covering the resist material 546 (block 448). The lift-off process severs an electrical connection between the two nanotip arrays 108 and 116 as depicted in FIG. 5H, leaving two separate electrical conductors 128 and 144 for each nanoelectrode array. In another embodiment, a direct etching process removes the section of the electrically conductive layer 548.

After formation of the wafer is cut into multiple nanoelectrode arrays, referred to as samples, according to methods known to the art (block 452). Process 400 continues by filling the cavities 540 in the section of the silicon wafer 106 and bottom silicon oxide layer 132 (block 456) in each sample. Electrical wires 136 and 148 are electrically connected to the electrically conductive metal layers 128 and 144, respectively, in the nanoelectrode arrays 108 and 116. As depicted in FIG. 5J, one embodiment of the fill process includes application of a conductive epoxy 120, such as a silver epoxy resin, followed by application of an electrically non-conductive elastomer or epoxy layer 124 that seals the nanoelectrode arrays 108 and 116. The electrically conductive epoxy 120 electrically and physically connects the electrical leads 136 and 148 to the conductors 128 and 144, respectively.

In some embodiments, process 400 concludes after block 456, and the two-terminal electrode array 110 in FIG. 5J can be used with the dielectric cap 524 in place. The metallic nanotips 112 extend through the cap 524, and the cap 524 protects the surface of each of the nanoelectrode arrays 108 and 116 during operation.

In another embodiment, process 400 applies a wet etchant to remove the cap layer 524 after depositing the metal layer to form the metallic nanotips 112 (block 460). The cap layer 524 may be removed either prior to or after filling the cavity 540 as described in block 456. As depicted in FIG. 1A, the nanoelectrode arrays 108 and 116 are fully exposed along the top surface of the two-terminal nanoelectrode array 100. The exposed surface promotes improved electrical contact with nerve tissue during monitoring and electrical stimulation procedures.

While process 400 is described in conjunction with fabrication of a two-terminal nanoelectrode array, process 400 can also be used to fabricate the three-terminal nanoelectrode array depicted in FIG. 1B and a variety of other electrode configurations that include nanotips to improve the SNR of detected electrical signals.

While the preferred embodiments have been illustrated and described in detail in the drawings and foregoing description, the same should be considered illustrative and not restrictive. For example, the electrode has been shown as an integrated electrode in which the first electrical contact and the second electrical contact are electrically isolated from one another within a single electrode. The two electrical contacts, each with an extending electrically conductive projection, could be formed in two separate electrodes and electrically connected to the same nerve to form a single electrically conductive path through the two electrodes and the nerve. All changes, modifications, and further applications are desired to be protected.

1. An electrode comprising:
a first electrical contact having at least one electrically conductive projection extending from a surface of the first electrical contact, the at least one electrically conductive projection being configured to engage tissue proximate to at least one nerve to enable the first electrical contact to electrically contact the nerve directly and form an electrically conductive path between the nerve and the electrical contact; and
a first electrical lead electrically connected to the first electrical contact to enable signals from the nerve to be received.

2. The electrode of claim 1 further comprising:
a second electrical contact having a second electrically conductive projection extending from a surface of the second electrical contact, the second electrically conductive projection being configured to engage the tissue proximate to the at least one nerve to enable the second electrical contact to electrically contact the nerve directly and form an electrically conductive path between the nerve and the second electrical contact, the first electrical contact and the second electrical contact being electrically isolated from one another; and
a second electrical lead electrically connected to the second electrical contact to enable an electrical path to be formed from the first electrical lead to the second electrical lead through the first electrical contact, the first electrically conductive projection, the nerve, the second electrical conductive projection, and the second electrical contact.

3. The electrode of claim 2 wherein each electrical contact includes a plurality of electrically conductive projections.

4. The electrode of claim 2 wherein the first electrical contact, the first electrically conductive projection, the second electrical conductive projection, and the second electrical contact are integrated in a single electrode.

5. The electrode of claim 3 wherein each of the plurality of electrically conductive projection has a height of less than 5 μm from the surface of each electrical contact.

6. The electrode of claim 1 further comprising:
a layer of electrically non-conductive material formed over the surface of the first contact pad and a first portion of the at least one projection, a second portion the at least one projection extending through the layer of non-metallic material.

7. The electrode of claim 6, the electrically non-conductive material essentially comprising silicon nitride.

8. The electrode of claim 2 further comprising:
a third electrical contact having a third electrically conductive projection extending from a surface of the third electrical contact, the third electrically conductive projection being configured to engage the tissue proximate to the at least one nerve to enable the third electrical contact to electrically contact the nerve directly and form an electrically conductive path between the nerve and the third electrical contact, the first electrical contact, second electrical contact, and the third electrical contact being electrically isolated from one another; and
a third electrical lead electrically connected to the third electrical contact to enable an electrical path to be formed from the first electrical lead to the third electrical lead through the first electrical contact, the first electrically conductive projection, the nerve, the third electrical conductive projection, and the third electrical contact.

9. The electrode of claim 8, the first electrical lead and third electrical lead being configured to conduct an electrical signal to stimulate the nerve.
